(19) United States
(12) Patent Application Publication
(10) Pub. No.: US 2019/0112655 A1
(43) Pub. Date: Apr. 18, 2019

(54) Method, Systems and Apparatus for High-Throughput Single-Cell DNA Sequencing With Droplet Microfluidics

(71) Applicant: Mission Bio, Inc., South San Francisco, CA (US)

(72) Inventors: Dennis Jay Eastburn, Burlingame, CA (US); Adam R. Sciambi, South San Francisco, CA (US); Maurizio Pellegrino, South San Francisco, CA (US)

(73) Assignee: Mission Bio, Inc., South San Francisco, CA (US)

(21) Appl. No.: 16/164,595

(22) Filed: Oct. 18, 2018

Related U.S. Application Data
(60) Provisional application No. 62/574,103, filed on Oct. 18, 2017.
(60) Provisional application No. 62/574,104, filed on Oct. 18, 2017.
(60) Provisional application No. 62/574,109, filed on Oct. 18, 2017.

(51) Int. Cl. C12Q 1/6874

(52) U.S. Cl. CPC C12Q 1/6874

(57) ABSTRACT

The disclosed embodiments relate to method, apparatus and system for high throughput single-cell DNA sequencing with droplet microfluidic. In an exemplary embodiment, a microfluidic apparatus is used to provide a rapid and cost-effective targeted genomic sequencing of thousands of cells in parallel. The targeted sequencing can be directed for residual disease detection. In one embodiment, the disclosure provides a method to detect one or more mutations in tumor cells, the method comprising: encapsulating at least one cell and a lysis reagent in a carrier fluid to form a droplet, wherein the cell originates from a tumor and the cell comprises a genomic DNA; lysing the cell to release the genomic DNA and thereby form a droplet containing the genomic DNA; introducing a cell identifier and one or more primers specific to a plurality of regions of the genomic DNA; and thermocycling the droplet to amplify the genomic DNA and to incorporate cell identifiers into the genomic DNA to produce a plurality of amplified DNA with identified loci; wherein once the cell identifier is incorporated into the amplified DNA, the identified loci are sequenced and at least one DNA mutation is identified for the tumor cells.

The instant application claims priority to U.S. Provisional Application Nos. 62/574,103 (filed Oct. 18, 2017), 62/574,104 (filed Oct. 18, 2017) and 62/574,109 (filed Oct. 18, 2017); the specification of each of which is incorporated herein in its entirety.

### BACKGROUND

The promise of precision medicine is to deliver highly targeted treatment to every single diseased cell. The conventional one-size-fits-all approach of medical treatments isn't working for many patients who need help. To move precision medicine forward, researchers and clinicians need to look at the origins of disease, the single cell, in new meaningful ways.

Because most diseases are not caused by just one mutation, understanding genetic variability, including mutation co-occurrence at the single-cell level, is vitally important for clinical researchers. This level of resolution is missed with existing bulk sequencing which can result in failed clinical trials, high costs, and poor patient outcomes. To impact precision drug discovery, development, and delivery, insight into the mutational differences within and among every single cell is needed.

The conventional technology for measuring cellular mutations and heterogeneity for complex disease is bulk sequencing based on averages. A problem with using averages is that the underlying genetic diversity is missed across cell populations. Understanding this diversity is important for patient stratification, therapy selection and disease monitoring. Moving beyond averages helps deliver on the promise of precision medicine.

Therefore, there is a need for method, system and apparatus to provide high-throughput, single-cell DNA sequencing.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 schematically a portion of an exemplary platform for implementing a first step of forming cell droplets according to one embodiment of the disclosure.

FIG. 2 schematically illustrates incubation of protease and cell droplets according to one embodiment of the disclosure.

FIG. 3 schematically illustrates bar coding of an exemplary droplet according to one embodiment of the disclosure.

FIG. 4 illustrates an exemplary process for implementing the disclosed principles.

FIG. 5A shows cell distribution for an application of the disclosed embodiments without protease (no protease).

FIG. 5B shows the resulting cell distribution for an application of the disclosed embodiments for a sample with protease.

FIG. 5C shows the NGC library yields and size distribution at 371 base pairs with and without protease from the sample of FIG. 5B.

FIG. 5D shows the percentage of barcode reads for the eight targeted genomic loci for a sample with protease and a sample without protease.

FIG. 6 shows tabulated results of a variant allele information of a targeted panel according to an exemplary implementation of the disclosure.

FIG. 7A is a table displaying key metrics from the diagnosis, remission and relapse single cell DNA sequencing run from an AML patient.

FIG. 7B shows the performance of the panel across the targeted loci for each of the three testing stages.

FIG. 8 shows the performance of the AML panel across the targeted locis of AML, genome tested according to the disclosed embodiments.

FIG. 9 is a table showing 17 different variant alleles identified in the AML patient samples.

FIG. 10 shows the presence of each of the 17 alleles of FIG. 9 in different sample populations (diagnosis, remission and relapse).

FIG. 11A shows diagnosis sample single-cell VAFs for each of the 4 non-synonymous mutations identified for the AML patient.

FIG. 11B shows the heat maps denoting single-cell genotypes for the three longitudinal AML patient samples. Non-patient Raji cells have been removed.

FIG. 11C shows the clonal populations identified from clinical bone marrow biopsies taken at the time for diagnosis, remission and relapse. Non-patient Raji cells have been removed.

FIG. 12 shows the comparative results for bulk VAFs versus VAFs acquired from the disclosed single-cell sequencing workflow when the barcode identifiers were removed.

FIG. 13 shows a comparison of single-cell sequencing data from the diagnosis sample obtained from our workflow and a simple clonal inference of the diagnosis cell clonal populations produced from the bulk VAFs. Non-patient Raji cells have been removed.

FIG. 14 is a table showing 295 genes that were targeted for bulk sequencing according to one embodiment of the disclosure.

### DETAILED DESCRIPTION

Current tumor sequencing paradigms are inadequate to fully characterize many instances of AML (acute myeloid leukemia). A major challenge has been the unambiguous identification of potentially rare and genetically heterogeneous neoplastic cell populations with subclones capable of critically impacting tumor evolution and the acquisition of therapeutic resistance. Conventional bulk population sequencing is often unable to identify rare alleles or definitively determine whether mutations co-occur within the same cell. Single-cell sequencing has the potential to address these key issues and transform our ability to accurately characterize clonal heterogeneity in AML.

An established approach for high-throughput single-cell sequencing uses molecular barcodes to tag the nucleic acids of individual cells confined to emulsion droplets. Although it is now feasible to perform single-cell RNA sequencing on thousands of cells using this type of approach, high-throughput single-cell DNA genotyping using droplet microfluidics has not been demonstrated on eukaryotic cells. This is primarily due to the challenges associated with efficiently lysing cells, freeing genomic DNA from chromatin and enabling efficient PCR amplification in the presence of high concentrations of crude lysate.

To overcome these and other shortcoming of the conventional systems and to enable the characterization of genetic diversity within cancer cell populations, an embodiment of the disclosure provides a microfluidic droplet workflow that enables efficient and massively-parallel single-cell PCR-based barcoding. The microfluidic droplet workflow may be implemented in one or more steps on one or more instruments.

As stated, an embodiment of the disclosure provides a system and platform for scalable detection of genomic variability within and across cell populations. In one embodiment, the platform includes an instrument, consumables and software, which connect seamlessly into an existing Next-Generation Sequencing (“NGS”) workflows. The disclosed platform provides a highly sensitive and customizable solution that is fully supported to enable biologically and clinically meaningful discoveries.

In one application of the disclosed embodiments, the platform utilizes a droplet microfluidic approach to identify heterogeneity in a population of at least 10,000 cells. Utilizing the disclosed droplet microfluidic embodiment allows rapid encapsulation, processing and profiling of thousands of individual cells for single-cell DNA applications. This enables accessing DNA for the detection of mutation co-occurrence at unprecedented scale. This approach also allows single nucleotide variant (“SNV”) and indel detection while maintaining low allele dropout and high coverage uniformity as compared to the conventional methods requiring whole genome amplification. The disclosed embodiments are capable of working with customized content. Thus, the focus may remain on the targets of intertest that are most informative for disease detection and research. The ability to understand cellular heterogeneity at the single-cell level helps drive precision medicine.

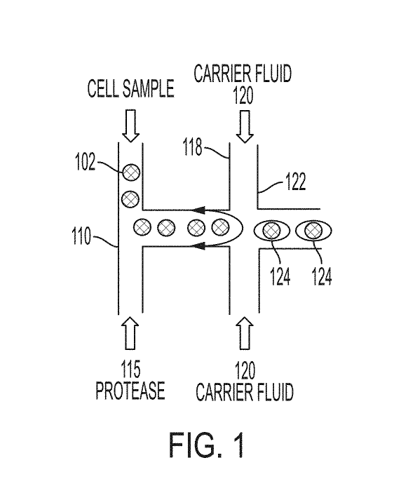

FIG. 1 schematically a portion of an exemplary platform for implementing a first step of forming cell droplets according to one embodiment of the disclosure. Specifically, FIG. 1 shows, among others, the steps of cell encapsulation by partitioning cells into individual droplets and adding protease to the droplets. In FIG. 1, cell samples 102 are introduced into tubing system 110. Cells 102 my originate from a tumor. Cells 102 may be collected at different stages. For example, cells 102 may be collected at diagnosis, remission or relapse.

The cells may be extracted from biological samples. As used herein, the phrase biological sample encompasses a variety of sample types obtained from an individual and can be used in a diagnostic or monitoring assay. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term biological sample encompasses a clinical sample, and also includes cells in culture, cell supernatants, cell lysates, cells, serum, plasma, biological fluid, and tissue samples. Further, Biological sample may include cells; biological fluids such as blood, cerebrospinal fluid, semen, saliva, and the like; bile; bone marrow; skin (e.g., skin biopsy); and antibodies obtained from an individual.

In various aspects the subject methods may be used to detect a variety of components from such biological samples. Components of interest include, but are not necessarily limited to, cells (e.g., circulating cells and/or circulating tumor cells), polynucleotides (e.g., DNA and/or RNA), polypeptides (e.g., peptides and/or proteins), and many other components that may be present in a biological sample.

“Polynucleotides” or “oligonucleotides” as used herein refer to linear polymers of nucleotide monomers, and may be used interchangeably. Polynucleotides and oligonucleotides can have any of a variety of structural configurations, e.g., be single stranded, double stranded, or a combination of both, as well as having higher order intra- or intermolecular secondary/tertiary structures, e.g., hairpins, loops, triple stranded regions, etc. Polynucleotides typically range in size from a few monomeric units, e.g., 5-40, when they are usually referred to as “oligonucleotides,” to several thousand monomeric units. Whenever a polynucleotide or oligonucleotide is represented by a sequence of letters (upper or lower case), such as “ATGCCTG”, it will be understood that the nucleotides are in 5′.fwdarw.3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, N.Y., 1999).

The terms “polypeptide”, “peptide”, and “protein”, used interchangeably herein, refer to a polymeric form of amino acids of any length. NH₂ refers to the free amino group present at the amino terminus of a polypeptide. COOH refers to the free carboxyl group present at the carboxyl terminus of a polypeptide. In keeping with standard polypeptide nomenclature, J. Biol. Chem., 243 (1969), 3552-3559 is used.

In certain aspects, methods are provided for counting and/or genotyping cells, including normal cells or tumor cells, such as CTCs. A feature of such methods is the use of microfluidics.

In some instances, cells 102 may comprise nucleic acids wherein the nucleic acids are from a tumor cell. In some instances, cells 102 may comprise a whole, intact cell. In some instances, droplet 102 may comprise a cell lysate. In some instances, a droplet comprises a partially lysed cell. In some instances, methods disclosed herein comprise lysing a cell before containing the nucleic acids thereof in a droplet.

In some instances, methods disclosed herein comprise lysing a cell after containing the nucleic acids thereof in a droplet. In some instances, methods comprise containing a cell and cell lysis reagents in a droplet. In some instances, methods comprise contacting a droplet with a cell lysis reagent. In some instances, methods comprise injecting a droplet with a cell lysis reagent. In some instances, methods comprise flowing droplets into a cell lysis reagent. In some instances, methods comprise flowing cell lysis reagent into a carrier fluid comprising droplets. In some instances, the lysis reagent comprises a detergent. In some instances, the lysis reagent comprises a protease. In some instances, the lysis reagent comprises a lysozyme. In some instances, the lysis reagent comprises a protease. In some instances, the lysis reagent comprises an alkaline buffer.

Encapsulating a component from a biological sample may be achieved by any convenient method. In one exemplary method, droplets are formed in a massively parallel fashion in a serial bisection device.

As shown in FIG. 1, protease 115 is introduced at a branch of tubing 110. Protease 115 may be used to solubilize cells 102. Protease 102 may comprise any conventional protease having one or more enzyme to perform proteolysis including protein catabolism by hydrolysis of peptide bonds.

At inlet 118, carrier fluid 120 is added to the mixture of cells 102 in protease 115. Adding carrier fluid 120 causes formation of droplets 124. Droplets 124 may generally contain cell 102 and protease 115. Droplets 124 are suspended in carrier fluid 120. Carrier fluid may comprise hydrogel or other material that is immiscible with protease 115 and cells 120.

In FIG. 1, a first microfluidic channel and a second microfluidic channel can join at a junction such that the first fluid and the immiscible carrier fluid can intersect to reliably generate a plurality of droplets 124. In one embodiment, the droplets may comprise cells 102 and protease 115.

In another embodiment, the droplets may be configured to additionally and optionally include cell lysates, nucleic acids of cells, solid supports (e.g., beads), barcode oligonucleotides, or a combination thereof. The immiscible carrier fluid 120 may segment the first fluid to generate the plurality of droplets 124. For example, the plurality of droplets 124 can be generated immediately or substantially immediately after the junction of the first microfluidic channel and the second microfluidic channel. Droplets 124 may be generated immediately or substantially immediately after the intersection of the first fluid and the immiscible carrier fluid. The droplets may be generated without any sorting steps. In some instances, methods comprise incorporating a solid support, e.g., a bead (not shown) into the droplets. Controllably generating droplets containing a solid support therein can facilitate controlled combination of the solid support with one or more components downstream. Non-limiting examples of components downstream are cells, cell lysis reagents, cell lysates, nucleic acids, and reagents for nucleic acid synthesis, such as a nucleic acid amplification process.

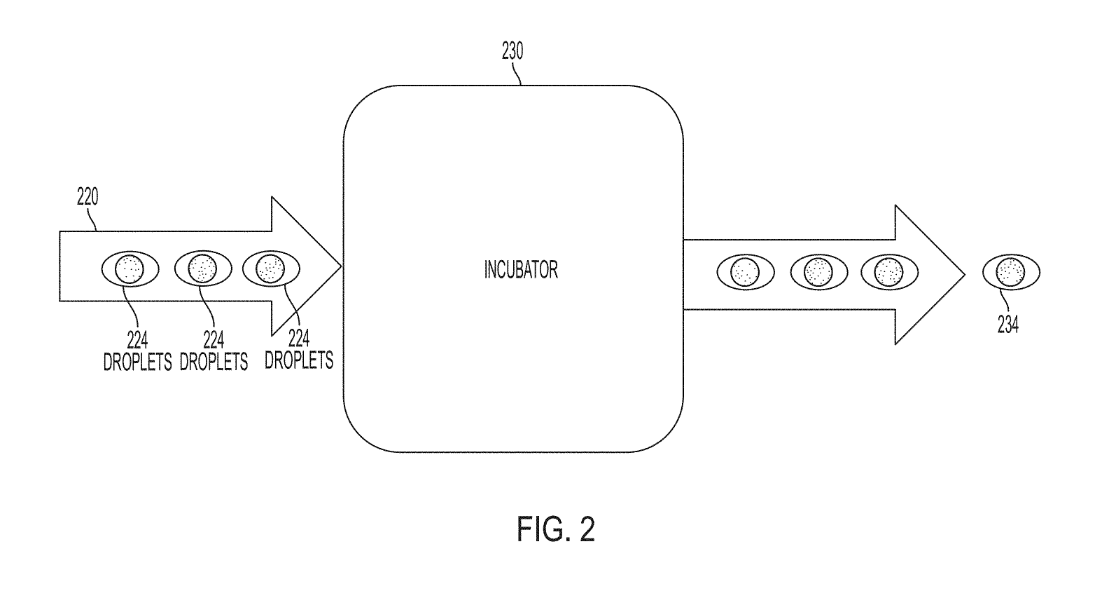

FIG. 2 schematically illustrates incubation of protease and cell droplets according to one embodiment of the disclosure. In one embodiment, the process shown in FIG. 2 can be considered as the lysate preparation process. In FIG. 2, droplets 224 (cell and protease droplets 124, FIG. 1) are directed to incubator 230. Incubator 230 provides cell lysis and protease digestion. Droplets 224 are suspended in oil stream 220 as in FIG. 1. In certain embodiments, incubator 230 may incubate a one or more temperatures (e.g., 50° C. and 80° C.) for one or more intervals.

The output of incubator 230 is lysate droplets 234. Lysate droplets 234 may be used for genomic DNA amplification. Following the lysate preparation, the protease in the droplet is inactivated by heat denaturation and each droplet containing genome of an individual cells is paired with a molecular bar code and PCR amplification reagent.

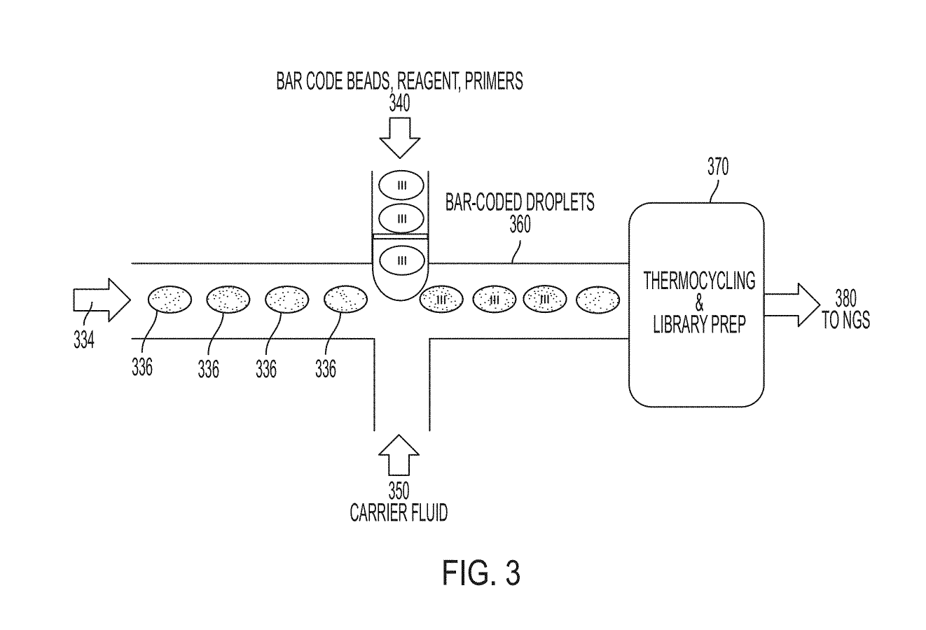

FIG. 3 schematically illustrates bar coding of an exemplary droplet according to one embodiment of the disclosure. In FIG. 3, stream 334 includes lysate droplets 336, substantially similar to the lysate droplet 234 of FIG. 2. In one embodiment, stream 340 may include bar code beads, reagent and primers. In one embodiment, carrier fluid 350 may be added. Second droplets 360 may comprise a cell identifier (e.g., barcode) and one or more primers specific to a plurality of regions of the genomic DNA. The primers may be designed and/or selected to target specific and desired regions of the genomic DNA.

In certain embodiments, barcoded droplets 340 may comprise bar-coded beads. As stated, one or more reagent may be introduced into the continuous stream 340. Stream 340 may comprise PCR primers and reagents designed for amplification. In one embodiment, specific regions of interest of the cell is amplified while tagging each amplicon with a unique cell barcode. This preserves the cell's identity and maturation profile.

In one embodiment, TaqMan™ PCR amplification reagent may be used. The resulting droplets 360 contain cell lysis, bar code and reagent mix. Droplets 360 are then thermo-cycled and library-prepped through instrument 370 to produce cell library 380. Cell library 380 may be subjected to NGS or further identification processing. The processes shown at FIGS. 1-3 provide a unique approach to profile SNVs and indel mutations at the single-cell level, deciphering the true cellular heterogeneity that defines a tumor sample.

The single-cell data enables direct assessment of clonal architecture with detection of mutation co-occurrence patterns. Rather than identifying variants that co-occur within a sub-clone from comparable bulk variant allele frequencies, single-cell resolution uncovers the true distribution of genotypes and their segregation pattern across subclones.

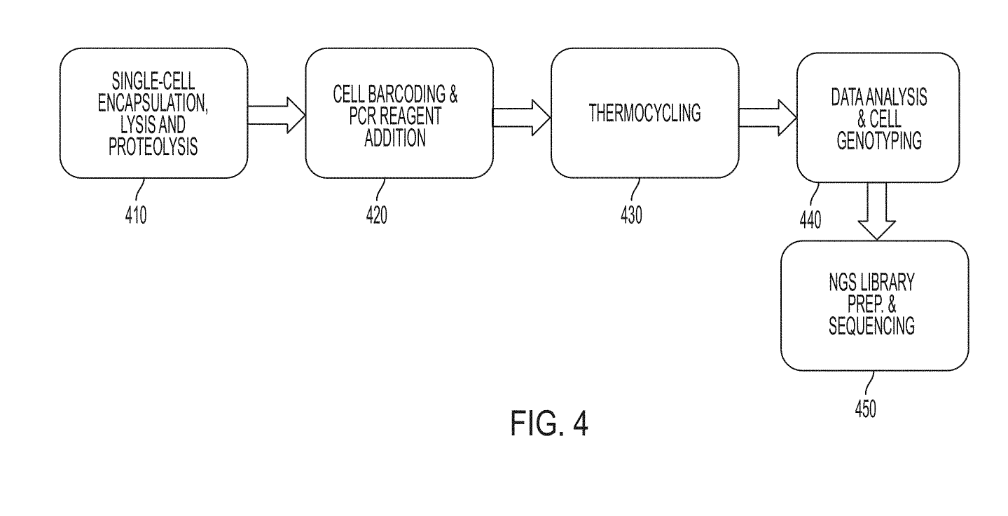

FIG. 4 illustrates an exemplary process for implementing the disclosed principles. The process of FIG. 4 starts at step 410 with single-cells encapsulation, lysis and proteolysis. Step 410 may be implemented with one or more sub-steps as described in references to FIGS. 1 and 2. At step 420, the encapsulated single-cell is bar-coded. One or more PCR reagent may also be added to the bar-coded single-cell. At step 430, the droplet containing the bar-coded single-cell with reagent is thermocycled to amplify the genome of interest. At step 440, the amplified cells are analyzed and the cells are genotyped. At step 450, NGS library prep and sequencing is performed to identify variants in the cell samples.

As stated, in certain embodiments, the microfluidic workflow first encapsulates individual cells in droplets, lyses the cells and prepares the lysate for genomic DNA amplification using proteases. In certain embodiments, following the lysate preparation step, the proteases are inactivated via heat denaturation and droplets containing the genomes of individual cells are paired with molecular barcodes and PCR amplification reagents.

Example 1—Protease based droplet workflow for single-cell genomic DNA amplification and barcoding. In this example, the process flow discussed in FIG. 4 was implemented on a group of cells. To demonstrate advantages of the protease in the two-step workflow, in one embodiment, droplet-based single-cell TaqMan™ PCR reactions were performed targeting the SRY locus on the Y chromosome, present as a single copy in a karyotypically normal cell. PCR-Activated Cell Sorting (“PACS”) were carried out on calcein violet stained DU145 prostate cancer cells encapsulated and lysed with or without the addition of a protease.

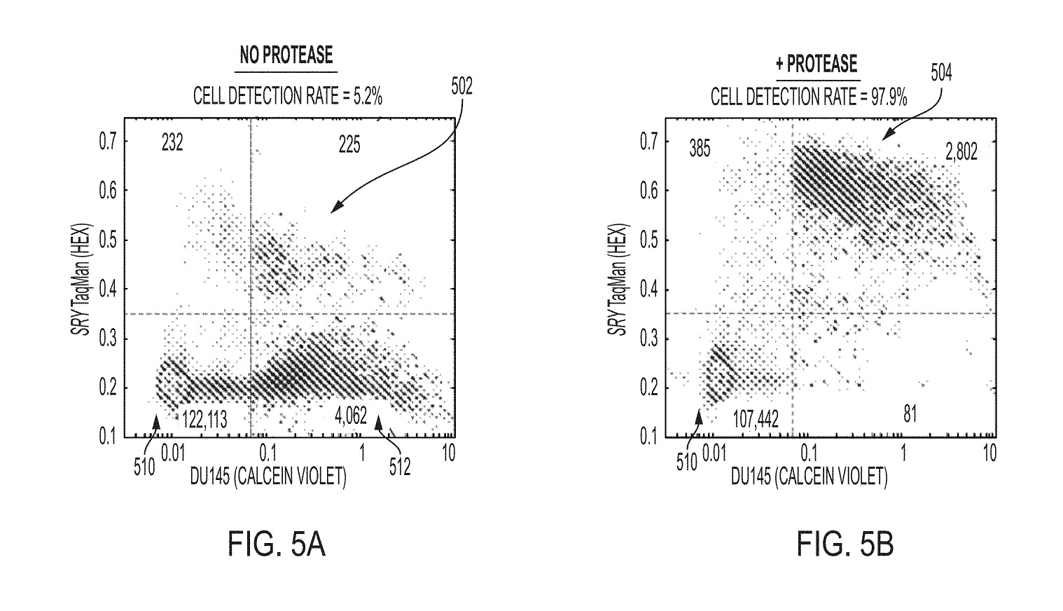

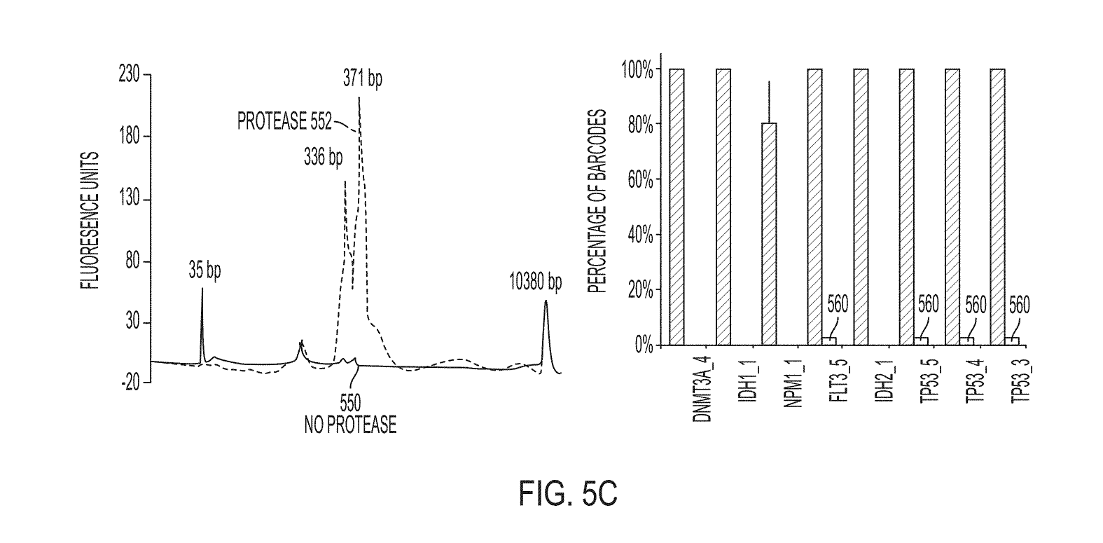

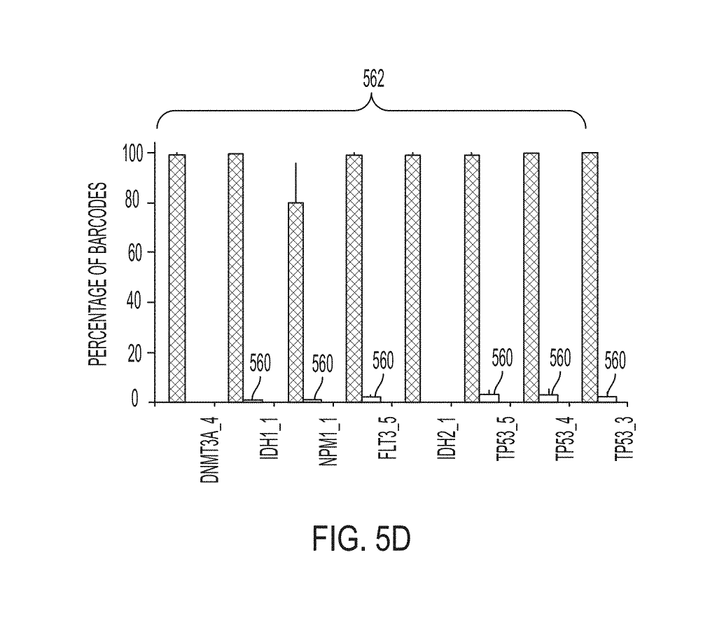

In the absence of protease during cell lysis, only 5.2% of detected DU145 cells were positive for TaqMan fluorescence. The inclusion of the protease resulted in a dramatically improved SRY locus detection rate of 97.9%. The results is shown in FIGS. 5A and 5B. In FIG. 5A, no protease was used and the denaturation rate was 5.2%. In FIG. 5B, protease was used and the denaturation rate of 97.9% was obtained.

More specifically, FIGS. 5A and 5B show the resulting cell distribution for an application of the disclosed embodiments for a sample with no-protease and a sample with protease. Here, cells (pseudo colored in blue (numbered 510 in FIGS. 5A and 5B)) were encapsulated with lysis buffer containing protease (yellow (numbered 512 in FIGS. 5A)) and incubated to promote proteolysis. Protease activity was then thermally inactivated and the droplets containing the cell lysate are paired and merged with droplets containing PCR reagents and molecular barcode-carrying hydrogel beads (pseudo colored in purple).

Next, the determination was made as to whether the two-step workflow was also required for single-cell barcoding of amplicons targeting 8 genomic loci located in TP53, DNMT3A, IDH1, IDH2, FLT3 and NPM1. To this end, hydrogel beads were synthesized with oligonucleotides containing both cell identifying barcodes and different gene specific primer sequences. These barcoded beads were microfluidically combined with droplets containing cell lysate generated with or without the protease reagent according to the disclosed process of FIG. 4.

Prior to PCR amplification, the oligonucleotides are photo-released from the hydrogel supports with UV exposure. Consistent with our earlier single-cell TaqMan™ reaction observations, amplification of the targeted genomic loci was substantially improved by use of a protease during cell lysis. Although similar numbers of input cells were used for both conditions, the use of protease enabled greater sequencing library DNA yields as assessed by a Bioanalyzer.

The results is shown in FIGS. 5C and 5D. Specifically, FIG. 5C shows the NGC library yields and size distribution at 371 base pairs. FIG. 5C shows that when protease enzyme was left out of the workflow for single-cell gDNA PCR in droplets, only −5% of DU145 cells (viability stained on the x-axis) were positive for SRY TaqMan reaction fluorescence (y-axis). Using protease during cell lysis 552 improves the DU145cell detection rate to −98% (points in upper right quadrant 550). Points in the plot represent droplets.

FIG. 5D shows the percentage of barcode reads for the eight targeted genomic loci. The results of FIG. 5D show bioanalyzer traces of sequencing libraries prepared from cells processed through the workflow with (black trace 562) or without (red trace 560) the use of protease indicates that PCR amplification in droplets is improved with proteolysis. The two-step workflow with protease enables better sequencing coverage depth per cell across the 8 amplified target loci listed on the x-axis.

Moreover, following sequencing, the average read coverage depth for the 8 targets from each cell was considerably higher when protease was used in the workflow. This data demonstrates the advantage of the two-step workflow for efficient amplification across different genomic loci for targeted single-cell genomic sequencing with molecular barcodes.

Example 2—Analysis of AML clonal architecture. Samples were obtained from a patient with AML at the times of diagnosis, remission and relapse. Having developed the core capability to perform targeted single-cell DNA sequencing, we next sought to apply the technology to the study of clonal heterogeneity in the context of normal karyotype AML.

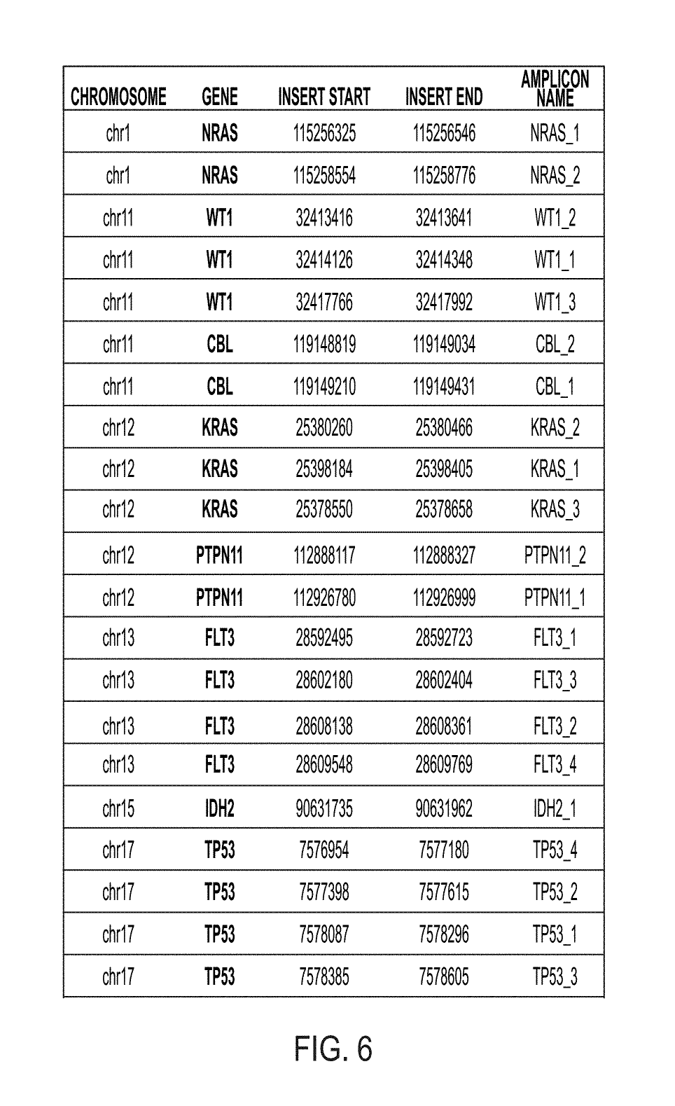

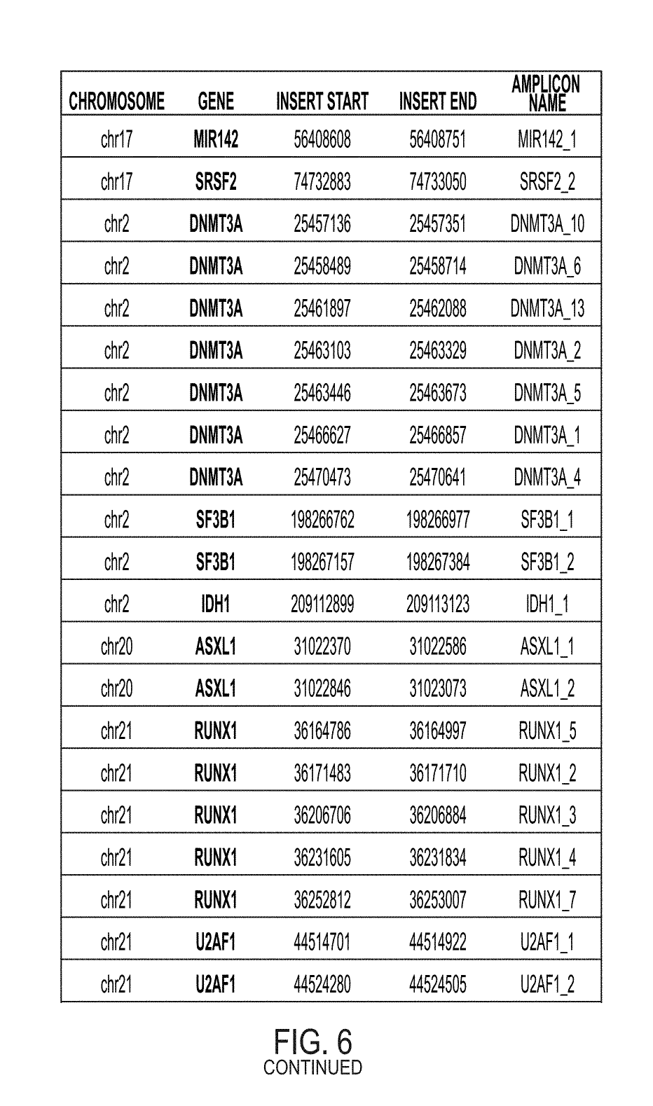

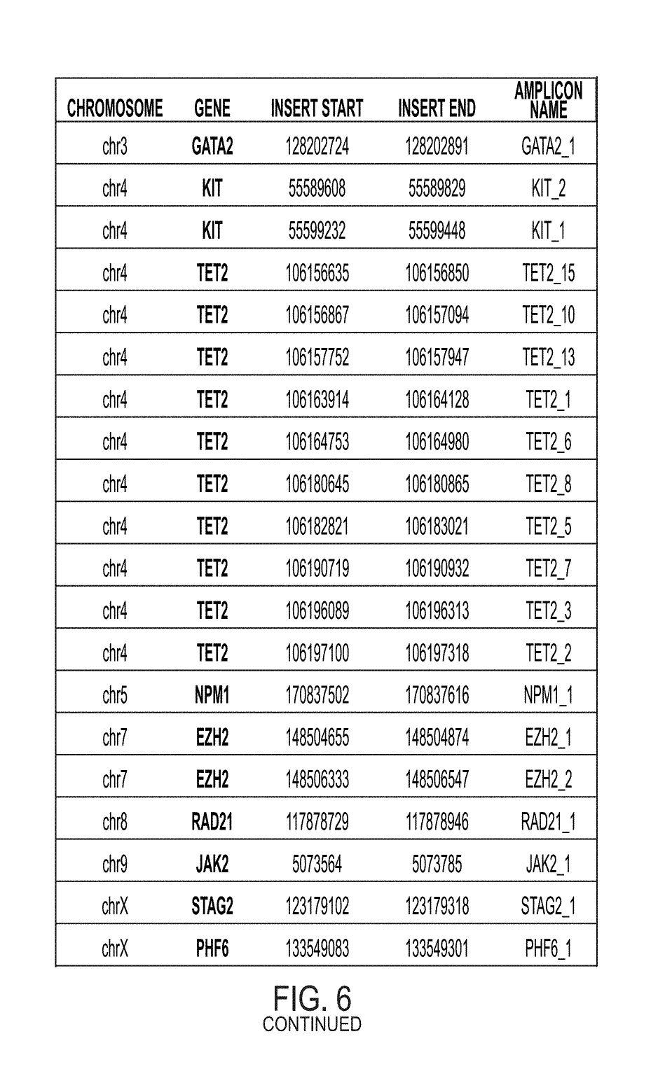

To provide variant allele information at clinically meaningful loci, we developed a 62 amplicon targeted panel that covers many of the 23 most commonly mutated genes associated with AML progression. The result is tabulated at Table 1 of FIG. 6. Following optimization for uniformity of amplification across the targeted loci (see Table 1), the panel was then used for single-cell targeted sequencing on AML patient bone marrow aspirates collected longitudinally at diagnosis, complete remission and relapse. Following thawing of frozen aspirates, the cells were quantified and immortalized Raji cells were added to the sample to achieve an approximate 1% spike in cell population. Known heterozygous SNVs within the Raji cells served as a positive control for cell type identification and a way to assess allele dropout in the workflow. Cell suspensions were then emulsified and barcoded with our workflow prior to bulk preparation of the final sequencing libraries. Total workflow time for each sample was less than two days. MiSeg™ runs generating 250 bp paired-end reads were performed for each of the three samples that were barcoded.

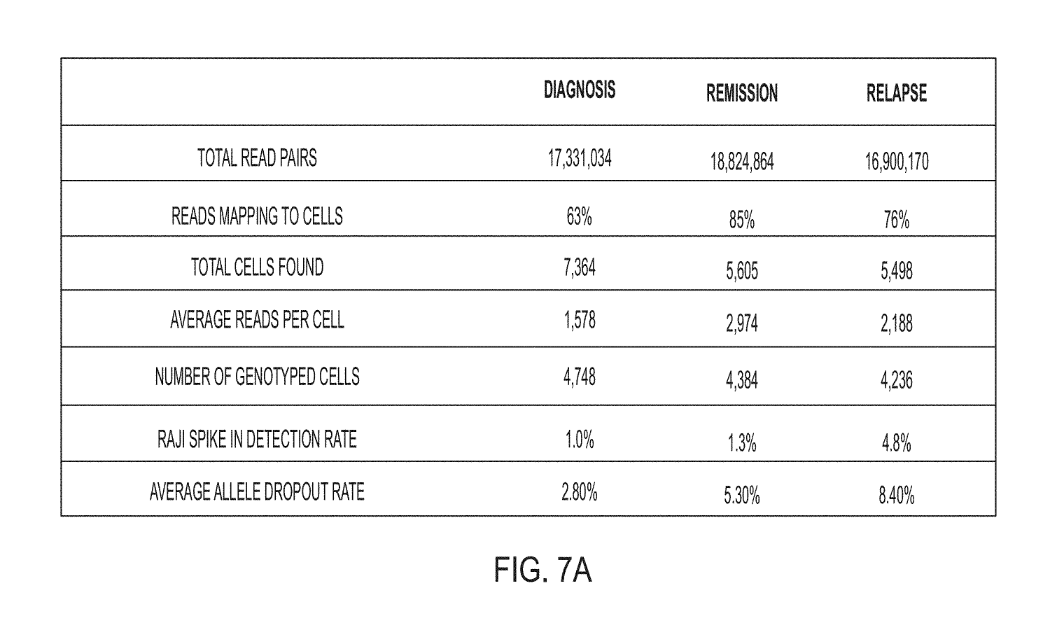

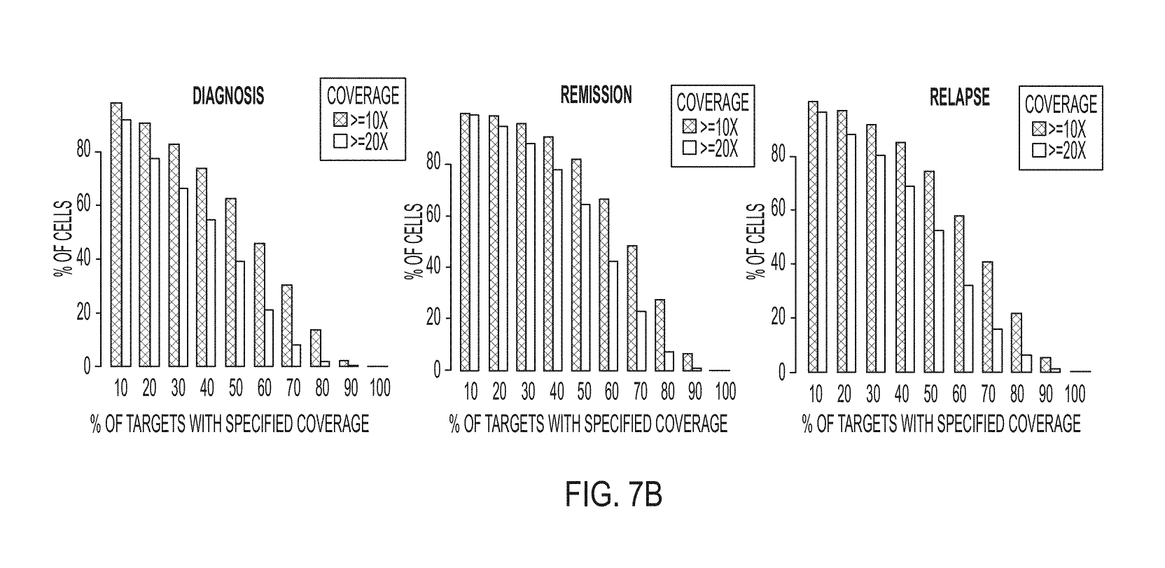

On average, 74.7% of the reads (MAPQ>30) were associated with a cell barcode and correctly mapped to one of the 62-targeted loci as shown in FIG. 7A. Specifically, FIG. 7A is a table displaying key metrics from the diagnosis, remission and relapse single cell DNA sequencing run from an AML patient.

Performance of the panel across the targeted loci is shown in FIG. 7B for each of the three stages of testing.

The Raji cell spike in detection rate across the three sample runs averaged 2.4% and the average allele dropout rate, calculated from two separate heterozygous TP53 SNVs present in the Raji cells, was 5.5% (see FIG. 7).

The allele dropout rate in FIG. 7 represents the percentage of cells within a run, averaged across the two loci, where the known heterozygous SNV was incorrectly genotyped as either homozygous wild type or homozygous mutant.

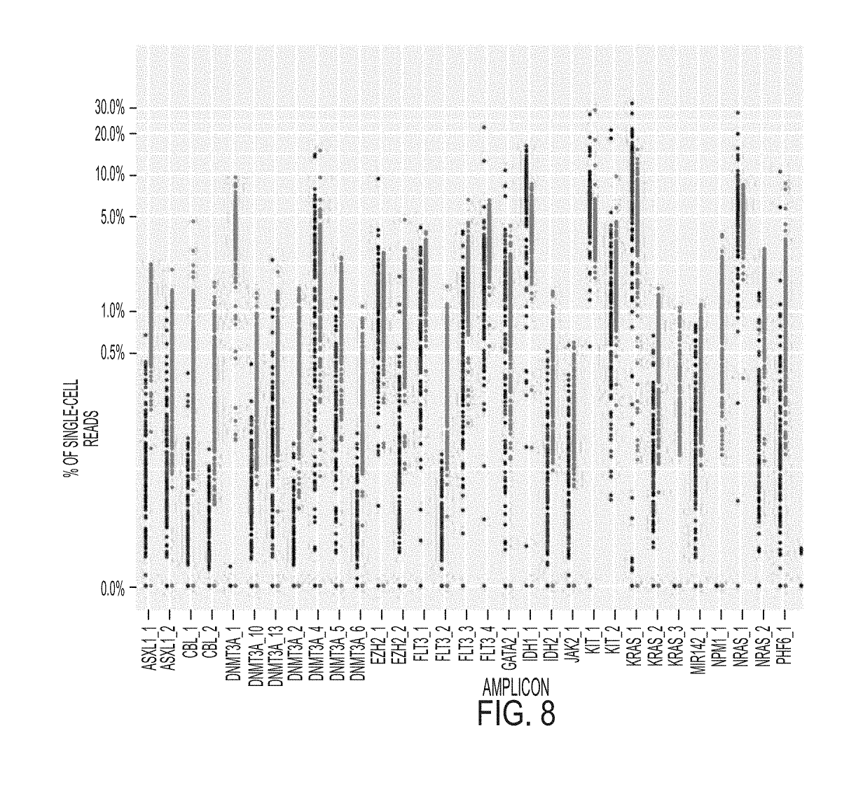

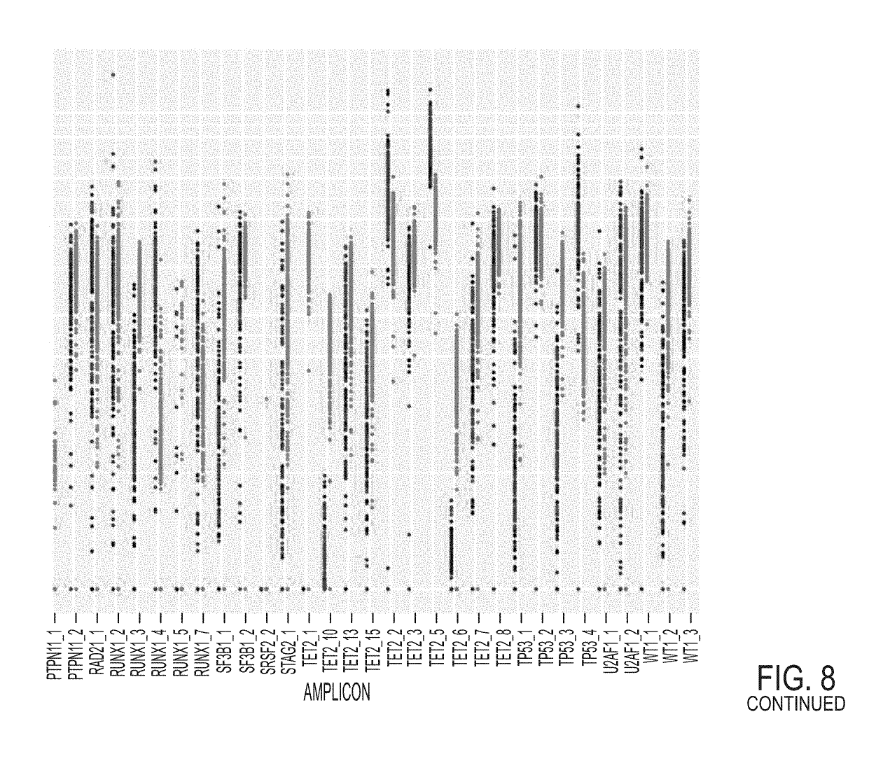

Performance of the AML panel across the targeted loci is shown in FIG. 8.

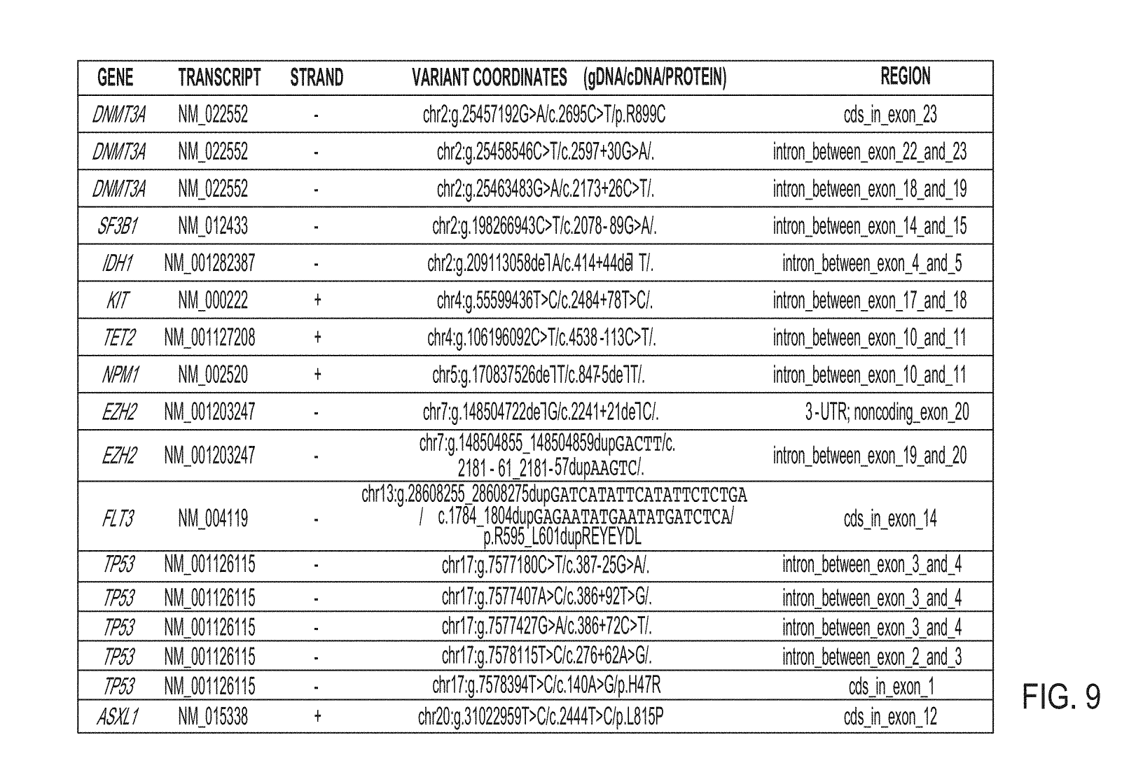

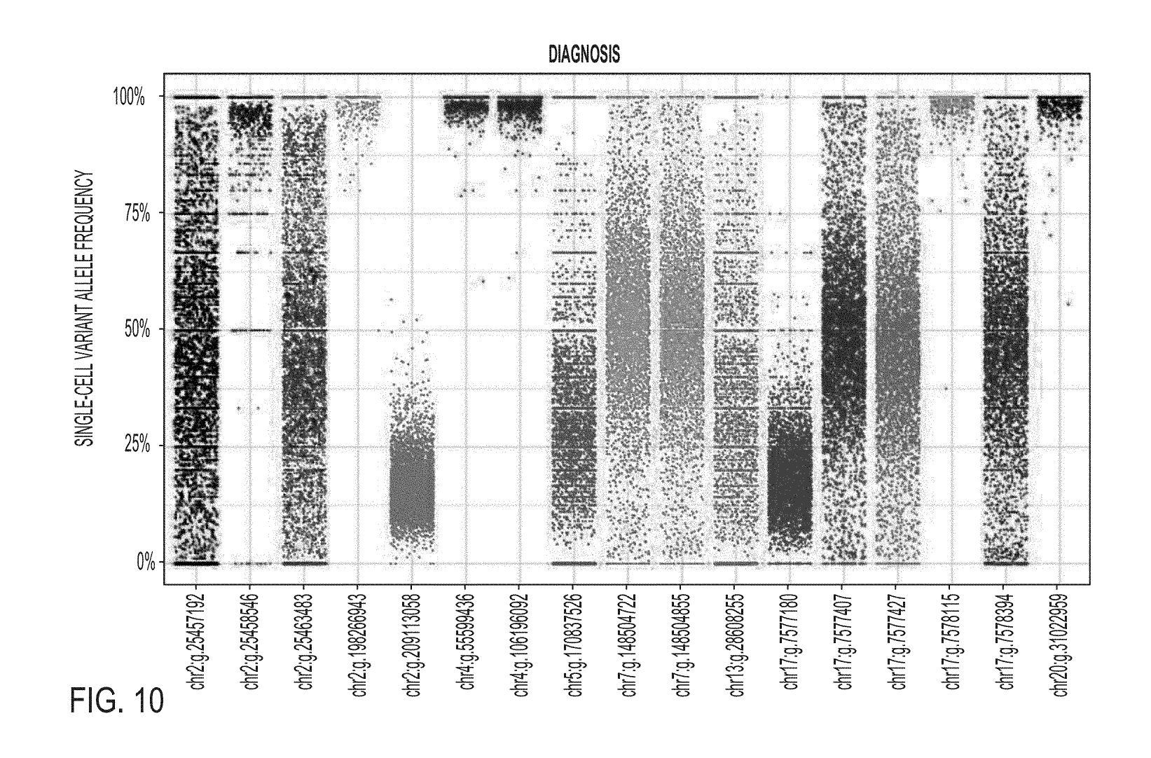

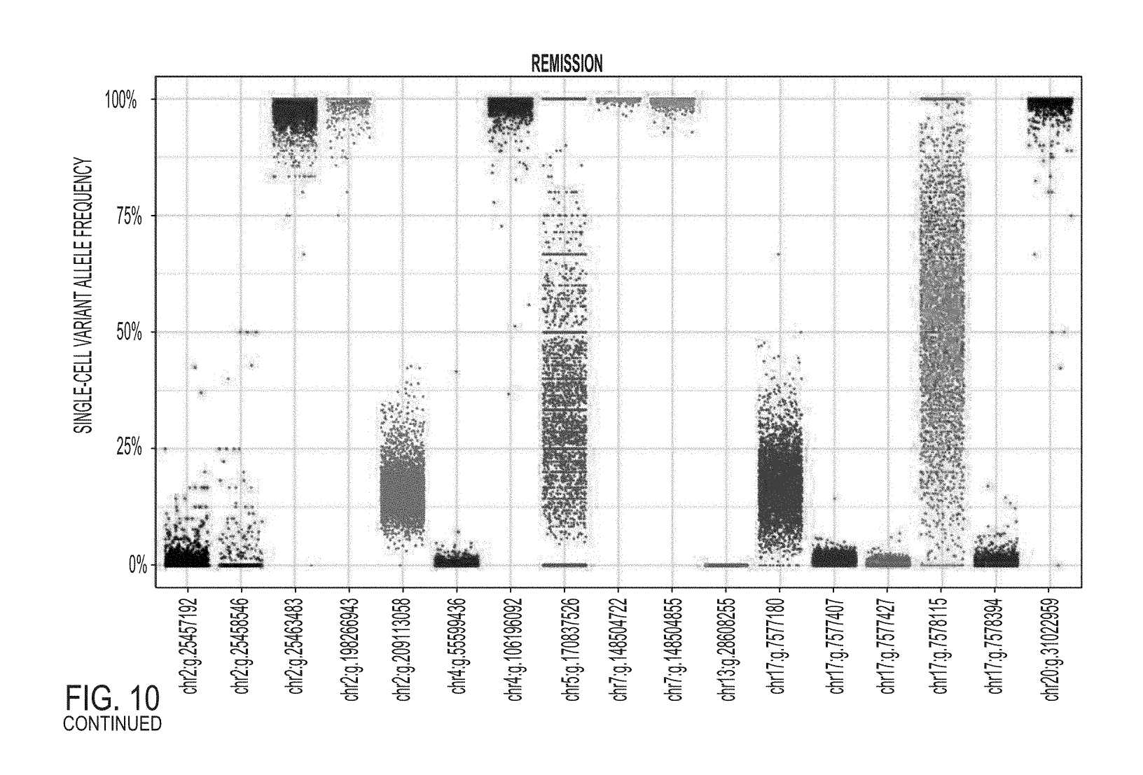

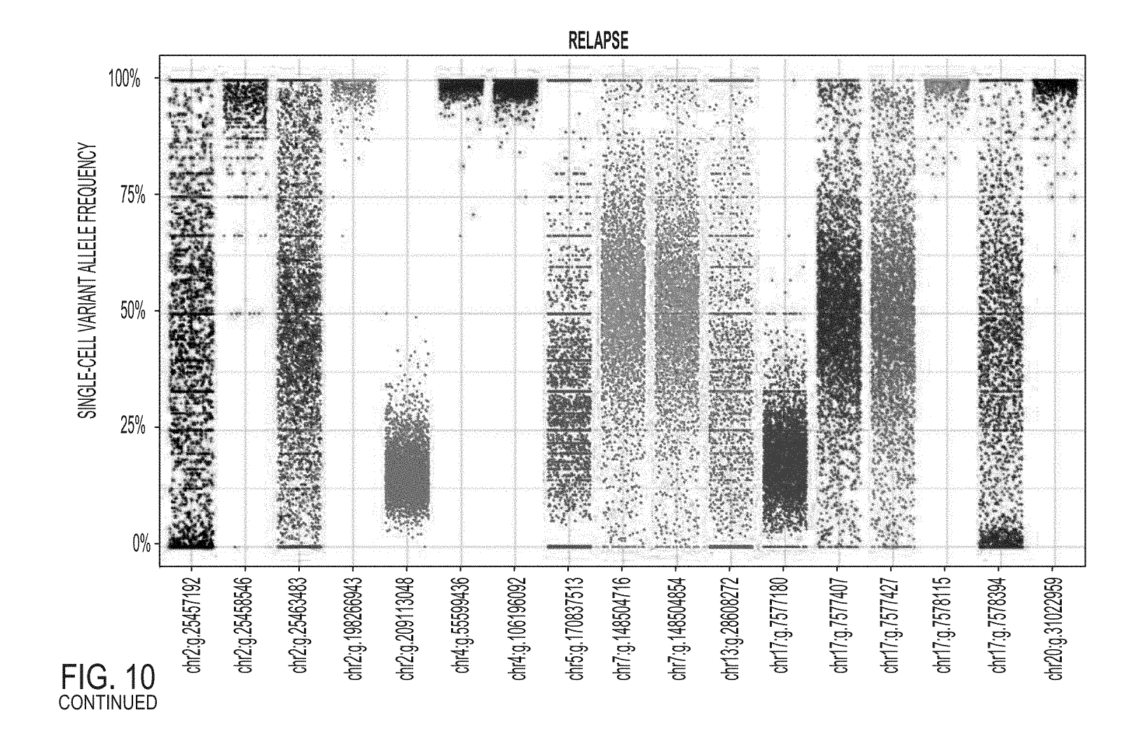

Using conventional genotype calling algorithms, a total of 17 variant alleles for this patient were identified. The identified alleles are shown at FIG. 9. FIG. 10 shows the presence of each of the 17 alleles of FIG. 9 in different sample populations (diagnosis, remission and relapse).

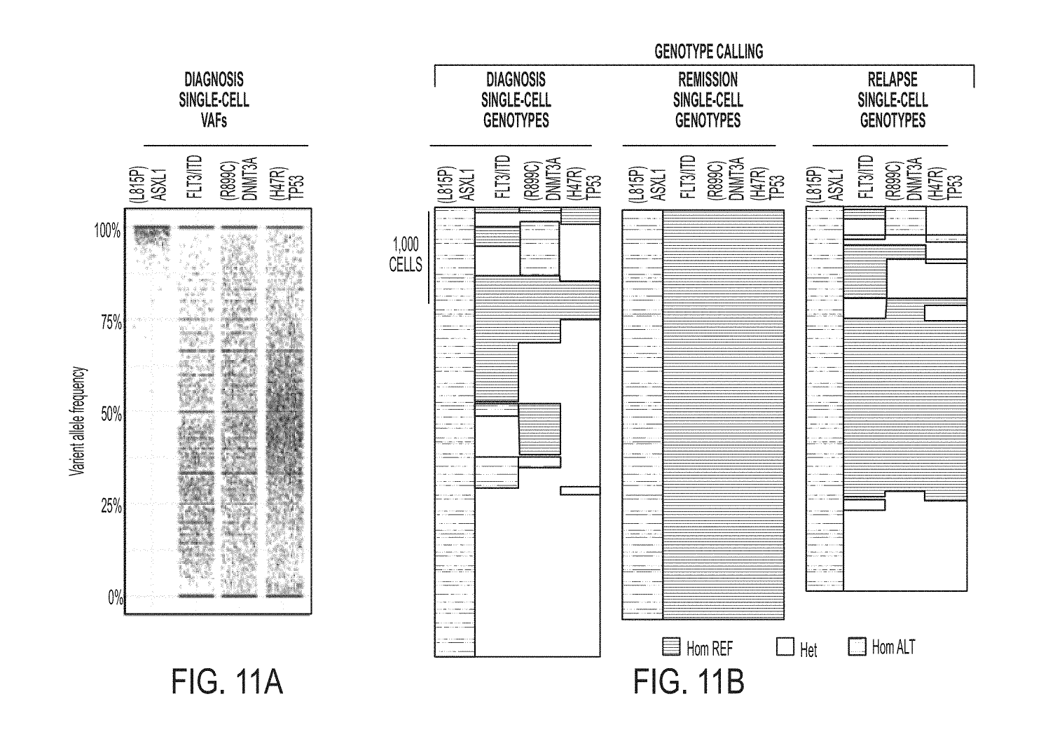

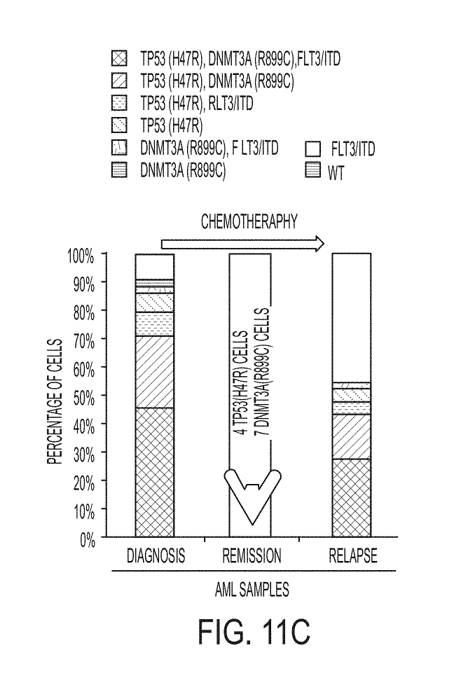

While 13 of these variants occurred in noncoding DNA, three non-synonymous SNVs were found in coding regions of TP53 (H47R), DNMT3A (R899C) and ASXL1 (L815P) from all three longitudinal samples. This is shown in FIGS. 11A, 11B and 11C.

FIG. 11A shows diagnosis sample single-cell VAFs for each of the 4 non-synonymous mutations identified for the AML patient. Here, the variant frequency of each allele is shown according to the shading.

FIG. 11B shows the heat maps denoting single-cell genotypes for the three longitudinal AML patient samples. The presence of a heterozygous alternate (ALT) allele is shown in red. Homozygous alternate alleles are shown in dark red and reference alleles are depicted in grey.

FIG. 11C shows the clonal populations identified from clinical bone marrow biopsies taken at the time of diagnosis, remission and relapse. Wild Type indicates cell that had reference genome sequence for TP53, DNMAT3A and FLT3, but were momozygous for the ASXL1 (L815P) mutation.

ASXL1 (L815P) is a previously reported common polymorphism (dbSNP: rs6058694) and was likely present in the germline since it was found in all cells throughout the course of the disease. Additionally, a 21 bp internal tandem duplication (ITD) in FLT3 was detected in cells from the diagnosis and relapse samples. FLT3/ITD alleles are found in roughly a quarter of newly diagnosed adult AML patients and are associated with poor prognosis. A total of 13,368 cells (4,456 cells per run average) were successfully genotyped at the four variant genomic loci (See FIGS. 7, 11A and 11B).

A comparison of the clonal populations from the diagnosis, remission and relapse samples indicates that the patient initially achieved complete remission, although having 10 mutant cells demonstrates the presence of minimal residual disease (“MRD”) at this time point (See FIG. 11C).

Despite the initial positive response to therapy, the reemergence of the clones present at diagnosis in the relapse sample indicates that it was ineffective at eradicating all of the cancer cells and, in this instance, did not dramatically remodel the initial clonal architecture of the tumor. Single-cell sequencing of additional cells from the remission sample may be required to test this hypothesis and identify additional MRD clones.

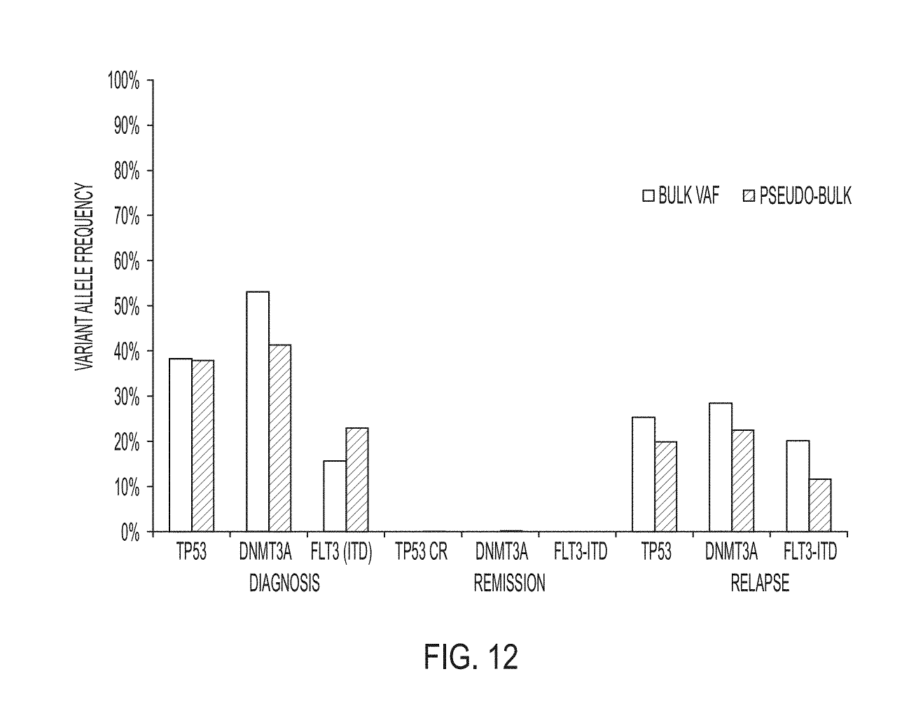

To assess the performance of the disclosed single-cell approach relative to conventional next generation sequencing (e.g., online methods, discussed below), bulk variant allele frequencies (VAFs) were obtained for the relevant mutations in two of the biopsy samples. The bulk VAFs were comparable to the VAFs acquired from the disclosed single-cell sequencing workflow (pseudo bulk VAFs) when the barcode identifiers are removed and the reads are analyzed in aggregate. The results are shown at FIG. 12.

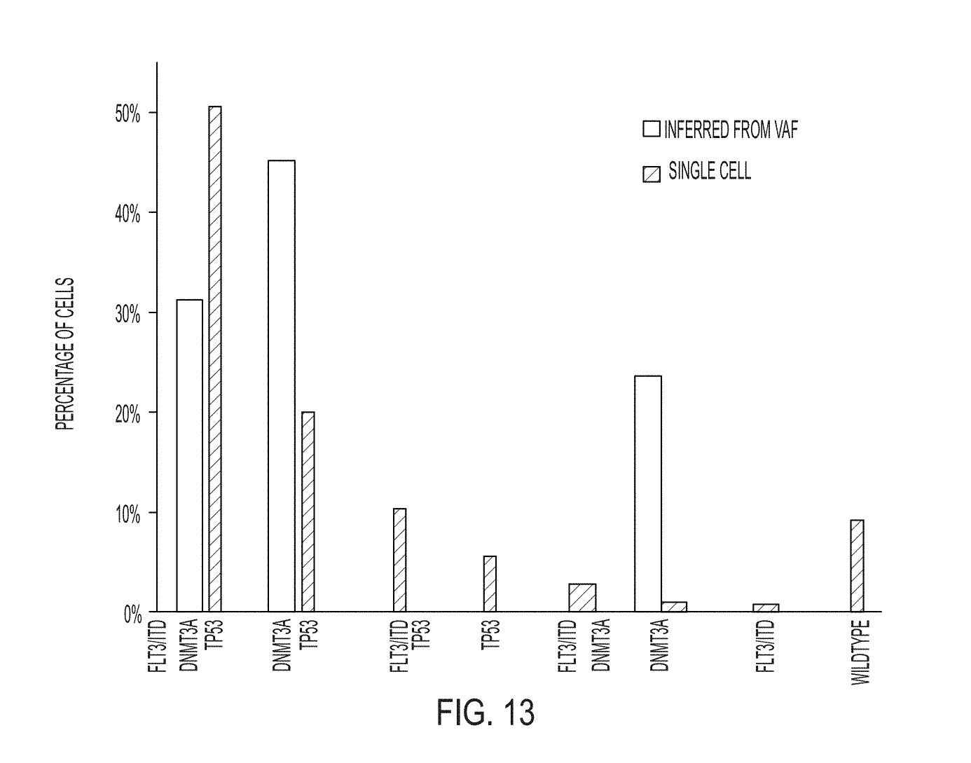

We next used the bulk sample VAFs to infer clonal architecture and compare it to the clonal populations obtained with our single-cell sequencing approach. The simplest model of inferred clonality predicts a significant DNMT3A (R899C) single mutant population indicative of founder mutation status (FIG. 13). FIG. 13 shows cells with greater than 20× read coverage of amplicon. This shows that disclosed workflow with protease enables better sequencing coverage depth per cell across the 8 amplified target loci listed on the x-axis.

Interestingly, the single-cell sequencing data does not support this model as only a relatively small DNMT3A single mutant population is observed and this population is at a frequency that can be explained by allele dropout. In contrast, our results suggest that the SNV in TP53 could be the founding mutation since the size of the TP53 (H47R) single mutant clone is larger than what would be expected from allele dropout. Our single-cell approach also unambiguously identified the TP53, DNMT3A and FLT3/ITD triple mutant population as the most abundant neoplastic cell type in the diagnosis and relapse samples (See 11C). Moreover, the identification of this clone strongly supports a model where the mutations were serially acquired during the progression of the disease.

As shown in Example 2, the disclosed embodiments provide rapid and cost-effective targeted genomic sequencing of thousands of AML cells in parallel which has not been feasible with conventional technologies. Applying the disclosed methods, system and apparatus to the study of larger AML patient populations will likely lead to correlations between clonal heterogeneity and clinical outcomes. Although the exemplary embodiments were focused on AML in this study, the disclosed principles are applicable to other cancer cell types and profiling of solid tumors that may have been dissociated into single-cell suspensions. This capability is poised to complement an increased scientific appreciation of the role that genetic heterogeneity plays in the progression of many cancers as well as a desire by clinicians to make personalized medicine a widespread reality.

The following provides additional information regarding certain implementation of the disclosed embodiments.

Online Methods—Cell and patient samples—Raji B-lymphocyte cells were cultured in complete media (RPMI 1640 with 10% fetal bovine serum (FBS), 100 U/ml penicillin, and 100 μg/ml streptomycin) at 37° C. with 5% CO2. Cells were pelleted at 400 g for 4 min and washed once with HBSS and resuspended in PBS that was density matched with OptiPrep (Sigma-Aldrich) prior to encapsulation in microfluidic droplets.

The clinical AML samples were obtained from a 66 year old man diagnosed with AML, French-American-British (FAB) classification M5. Pre-treatment diagnostic bone marrow biopsy showed 80% myeloblast and cytogenetic analysis showed normal male karyotype. The patient received an induction chemotherapy consisted of fludarabine, cytarabine and idarubicin. Day 28 bone marrow aspiration showed morphological complete remission (CR). The patient received additional 2 cycles of consolidation therapy with the same combination but approximately 3 months after achieving CR, his AML relapsed with 48% blast. The patient was subsequently treated with azacitidine and sorafenib chemotherapy and achieved second CR. The patient then underwent allogeneic stem cell transplant from his matched sibling but approximately 2 months after transplant, the disease relapsed. The patient was subsequently treated with multiple salvage therapies but passed away from leukemia progression approximately 2 years from his original diagnosis. Bone marrow from original diagnosis, first CR, and first relapse were analyzed. Patient samples were collected under an IRB approved protocol and patients singed the consent for sample collection and analysis. The protocol adhered to the Declaration of Helsinki.

Frozen bone marrow aspirates were thawed at the time of cell encapsulation and resuspended in 5 ml of FBS on ice, followed by a single wash with PBS. All cell samples were quantified prior to encapsulation by combining 5 μl aliquots of cell suspension with an equal amount of trypan blue (ThermoFisher), then loaded on chamber slides and counted with the Countess Automated Cell Counter (ThermoFisher). The Raji cells were added to the bone marrow cell samples to achieve a ˜1% final spike-in concentration.

Fabrication and operation of microfluidic device—A microfluidic device was constructed consistent with the disclosed principles. The microfluidic droplet handling on devices were made from polydimethylsiloxane (PDMS) molds bonded to glass slides; the device channels were treated with Aquapel to make them hydrophobic. The PDMS molds were formed from silicon wafer masters with photolithographically patterned SU-8 (Microchem) on them. The devices operated primarily with syringe pumps (NewEra), which drove cell suspensions, reagents and fluorinated oils (Novec 7500 and FC-40) with 2-5% PEG-PFPE block-copolymer surfactant into the devices through polyethylene tubing. Merger of the cell lysate containing droplets with the PCR reagent/barcode bead droplets was performed using a microfluidic electrode.

Generation of barcode containing beads—Barcoded hydrogel beads were made as previously reported in Klein et al. Briefly, a monomeric acrylamide solution and an acrydite-modified oligonucleotide were emulsified on a dropmaker with oil containing TEMED. The TEMED initiates polymerization of the acrylamide resulting in highly uniform beads. The incorporated oligonucleotide was then used as a base on which different split-and-pool generated combinations of barcodes were sequentially added with isothermal extension. Targeted gene-specific primers were phosphorylated and ligated to the 5′ end of the hydrogel attached oligonucleotides. ExoI was used to digest non-ligated barcode oligonucleotides that could otherwise interfere with the PCR reactions. Because the acrydite oligo also has a photocleavable linker (required for droplet PCR), barcoded oligonucleotide generation could be measured. We were able to convert approximately 45% of the base acrydite oligonucleotide into full-length barcode with gene specific primers attached. Single bead sequencing of beads from individual bead lots was also performed to verify quality of this reagent.

Cell encapsulation and droplet PCR—Following density matching, cell suspensions were loaded into 1 ml syringes and co-flowed with an equal volume of lysis buffer (100 mM Tris pH 8.0, 0.5% IGEPAL, proteinase K 1.0 mg/ml) to prevent premature lysing of cells3. The resultant emulsions were then incubated at 37° C. for 16-20 hours prior to heat inactivation of the protease.

Droplet PCR reactions consisted of 1× Platinum Multiplex PCR Master Mix (ThermoFisher), supplemented with 0.2 mg/ml RNAse A. Prior to thermocycling, the PCR emulsions containing the barcode carrying hydrogel beads were exposed to UV light for 8 min to release the oligonucleotides. Droplet PCR reactions were thermocycled with the following conditions: 95° C. for 10 min, 25 cycles of 95° C. for 30 s, 72° C. for 10 s, 60° C. for 4 min, 72° C. for 30 s and a final step of 72° C. for 2 min. Single-cell TaqMan reactions targeting the SRY locus were performed as previously described.

DNA recovery and sequencing library preparation—Following thermocycling, emulsions were broken using perfluoro-1-octanol and the aqueous fraction was diluted in water. The aqueous fraction was then collected and centrifuged prior to DNA purification using 0.63× of SPRI beads (Beckman Coulter). Sample indexes and Illumina adaptor sequences were then added via a 10 cycle PCR reaction with 1× Phusion High-Fidelity PCR Master Mix. A second 0.63× SPRI purification was then performed on the completed PCR reactions and samples were eluted in 10 μl of water. Libraries were analyzed on a DNA 1000 assay chip with a Bioanalyzer (Agilent Technologies), and sequenced on an Illumina MiSeq with either 150 bp or 250 bp paired end multiplexed runs. A single sequencing run was performed for each barcoded single-cell library prepared with our microfluidic workflow. A 5% ratio of Phi× DNA was used in the sequencing runs.

Analysis of next generation sequencing data—Sequenced reads were trimmed for adapter sequences (cutadapt), and aligned to the hg19 human genome using bwa-mem after extracting barcode information. After mapping, on target sequences were selected using standard bioinformatics tools (samtools), and barcode sequences were error corrected based on a white list of known sequences. The number of cells present in each tube was determined based on curve fitting a plot of number of reads assigned to each barcode vs. barcodes ranked in decreasing order, similar to what described in Macosko et. al. The total number of cells identified in this manner for a given sample run are presented in FIG. 7 as “Total cells found”. A subset of these cells was then identified that had sufficient sequence coverage depth to call genotypes at the 4 non-synonymous variant positions identified in TP53, ASXL1, FLT3 and DNMT3A. This subset of cells is presented as “Number of genotyped cells” in FIG. 7.

GATK 3.7¹¹ was used to genotype the diagnosis sample with a joint-calling approach. Mutations with a quality score higher than 8,000 were considered accurate variants. The presence of these variants as well as the potential FLT3/ITD were called at a single cell level across the three samples using Freebayes¹². TP53, ASXL1, FLT3 and DNMT3A genotype cluster analysis was performed using heatmap3 for R¹³. The non-patient Raji cell spike in populations were removed for this analysis.

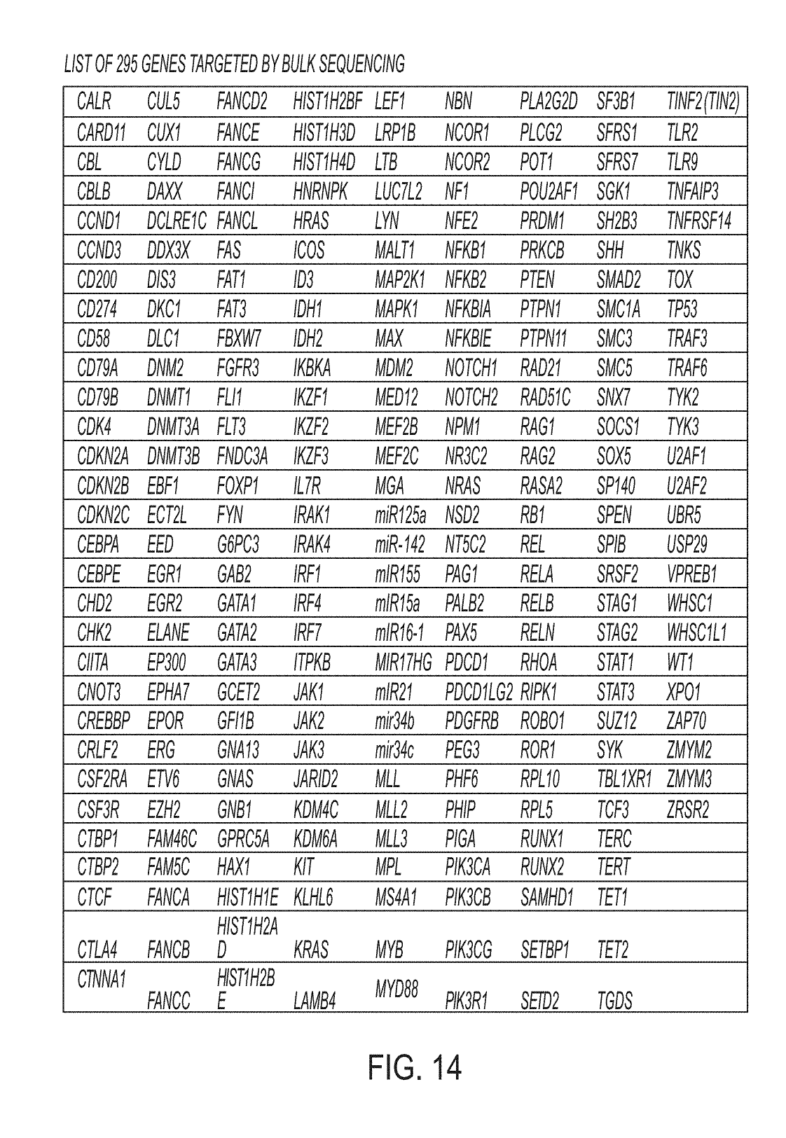

Bulk sequencing using capture targeted sequencing—We designed a SureSelect™ custom panel of 295 genes (Agilent Technologies, Santa Clara, Cailf.) that are recurrently mutated in hematologic malignancies (See FIG. 14). Extracted genomic DNA from bone marrow aspirates was fragmented and bait-captured according to manufacturer protocols. Captured DNA libraries were then sequenced using a HiSeq™2000 sequencer (Illumina, San Diego, Calif.) with 76 basepair paired-end reads.

The following examples are presented to further illustrates different embodiments of the disclosure. These examples are non-limiting and illustrative.

Example 1 is directed to a method to detect one or more mutations in tumor cells, the method comprising: encapsulating at least one cell and a lysis reagent in a carrier fluid to form a droplet, wherein the cell originates from a tumor and the cell comprises a genomic DNA; lysing the cell to release the genomic DNA and thereby form a droplet containing the genomic DNA; introducing a one or more cell identifiers and one or more primers specific to a plurality of regions of the genomic DNA; and thermocycling the droplet to amplify the plurality of regions of genomic DNA and to incorporate the one or more cell identifiers thereby producing amplified. DNA with the cell identifiers; wherein once the cell identifier is incorporated into the amplified DNA, the amplified regions are sequenced and at least one DNA mutation is identified for the tumor cells.

Example 2 is directed to the method of example 1, wherein a plurality of DNA mutations are identified for the tumor cells.

Example 3 is directed to the method of example 1, wherein the plurality of DNA mutations are identified substantially simultaneously for the tumor cells.

Example 4 is directed to the method of example 1, wherein the cell identifier is an oligonucleotide that serves as a cell barcode.

Example 5 is directed to the method of example 1, wherein the specific primers target 5-500 loci on the genomic DNA. In one embodiment, the specific primers target 10 or more loci on the genomic DNA.

Example 5 is directed to the method of example 1, wherein the specific primers target 10-500 loci on the genomic DNA. In one embodiment, the specific primers target 10-2,000 loci on the genomic DNA.

Example 6 is directed to the method of example 1, wherein the specific primers target 500-20,000 loci on the genomic DNA. In one embodiment, the specific primers target 500-2,000 loci on the genomic DNA.

Example 7 is directed to the method of example 1, wherein the lysis reagent comprises a protease.

Example 8 is directed to the method of example 1, wherein the specific primers target 2,000-100,000 loci on the genomic DNA.

Example 9 is directed to the method of example 1, wherein the number of tumor cells analyzed are about 10-1,000. In one embodiment, the number of tumor cells analyzed are about 100-1,000,000. In another embodiment, the detected mutation defines at least one attribute that correlates to a known disease.

Example 10 is directed to the method of example 1, wherein the number of tumor cells analyzed are about 1,000-100,000. In another embodiment, the number of tumor cells analyzed are about 10-100,000.

Example 11 is directed to the method of example 1, wherein the number of tumor cells analyzed are about 100,000-1,000,000.

Example 12 is directed to the method of example 1, wherein the detected mutation defines at least one attribute that correlates to a known disease.

Example 13 is directed to the method of example 1, wherein presence of the mutated cell is prognostic of a disease relapse.

Example 14 is directed to the method of example 1, wherein the at least one cell originates from a patient in disease remission.

Example 15 is directed to a method to detect one or more mutations in cells, the method comprising: forming a first droplet in a carrier fluid, the droplet having a tumor cell; lysing the tumor cell and releasing the genomic DNA to provide a released genomic DNA; forming a second droplet, the second droplet having the released genomic DNA. one or more cell identifier and one or more primers specific to a plurality of regions of the genomic DNA; and thermocycling the second droplet to amplify the plurality of regions of genomic DNA and to incorporate the one or more cell identifiers thereby producing; amplified DNA with cell identifiers; wherein once the one or more cell identifiers are incorporated into the amplified. DNA and wherein the amplified regions are sequenced and at least one DNA mutation is identified for the tumor cells.

Example 16 is directed to the method of example 15, wherein a plurality of DNA mutations are identified for the tumor cells.

Example 17 is directed to the method of example 15, wherein the plurality of DNA mutations are identified substantially simultaneously for the tumor cells.

Example 18 is directed to the method of example 15, wherein the specific primers target 10 or more loci on the genomic DNA.

Example 19 is directed to the method of example 15, wherein the specific primers target 10-500 loci on the genomic DNA. In one embodiment, the specific primers target 5 or more loci on the genomic DNA.

Example 20 is directed to the method of example 15, wherein the specific primers target 500-2,000 loci on the genomic DNA.

Example 21 is directed to the method of example 15, wherein the specific primers target 2,000-100,000 loci on the genomic DNA.

Example 22 is directed to the method of example 15, wherein the lysis reagent comprises a protease.

Example 23 is directed to the method of example 15, wherein the number of tumor cells analyzed are about 10-1,000.

Example 24 is directed to the method of example 15, wherein the number of tumor cells analyzed are about 1,000-100,000

Example 25 is directed to the method of example 15, wherein the number of tumor cells analyzed are about 100,000-1,000,000

Example 26 is directed to the method of example 15, wherein the detected mutation defines at least one attribute that correlates to a known disease.

Example 27 is directed to the method of example 15, wherein presence of the mutated cell is prognostic of a disease relapse.

Example 28 is directed to the method of example 15, wherein the at least one cell originates from a patient in disease remission.

Example 29 is directed to a system to detect one or more mutations in tumor cells, comprising: a first microfluidic channel to encapsulate at least one cell and a lysis reagent in a carrier fluid to form a droplet, wherein the cell originates from a tumor; an incubator to lyse the cell to release the genomic DNA and thereby form a droplet containing the genomic DNA; a second microfluidic channel to introduce a cell identifier and one or more primers specific to a plurality of regions of the genomic DNA to the droplet; and a thermocycler to thermocycle the droplet to amplify the genomic DNA and to incorporate cell identifiers into the genomic DNA to thereby produce a plurality of amplified DNA with identified loci; wherein once the cell identifier is incorporated into the amplified DNA, the identified loci are sequenced and at least one DNA mutation is identified for the tumor cells.

Example 30 is directed to the system of example 29, wherein a plurality of DNA mutations are identified for the tumor cells.

Example 31 is directed to the system of example 29, wherein the plurality of DNA mutations are identified substantially simultaneously for the tumor cells.

Example 32 is directed to the system of example 29, wherein the specific primers target 10 or more loci on the genomic DNA.

Example 33 is directed to the system of example 29, wherein the specific primers target 10-500 loci on the genomic DNA.

Example 34 is directed to the system of example 29, wherein the specific primers target 500-2,000 loci on the genomic DNA.

Example 35 is directed to the system of example 29, wherein the specific primers target 2,000-100,000 loci on the genomic DNA.

Example 36 is directed to the system of example 29, wherein the lysis reagent comprises a protease.

Example 37 is directed to the system of example 29, wherein the number of tumor cells analyzed are about 10-1,000.

Example 38 is directed to the system of example 29, wherein the number of tumor cells analyzed are about 1,000-100,000

Example 39 is directed to the system of example 29, wherein the number of tumor cells analyzed are about 100,000-1,000,000.

Example 40 is directed to the system of example 29, wherein the detected mutation defines at least one attribute that correlates to a known disease.

Example 41 is directed to the system of example 29, wherein presence of the mutated cell is prognostic of a disease relapse.

Example 42 is directed to the system of example 29, wherein the at least one cell originates from a patient in disease remission.

Example 43 is directed to a system to detect one or more mutations in cells, comprising: a first microfluidic channel to form a first droplet in a carrier fluid, the droplet having a tumor cell; an incubator to lyse the tumor cell and to release the genomic DNA; a second microfluidic channel to form a second droplet, the second droplet having a cell identifier and one or more primers specific to a plurality of regions of the genomic DNA; and a thermocycler to thermocycle the second droplet to amplify the genomic DNA and to incorporate the identifier into the genomic DNA to thereby produce a plurality of amplified DNA with identified loci; wherein once the cell identifier is incorporated into the amplified DNA, the identified loci are sequenced and at least one DNA mutation is identified for the tumor cells.

Example 44 is directed to the system of example 43, wherein a plurality of DNA mutations are identified for the tumor cells.

Example 45 is directed to the system of example 43, wherein the plurality of DNA mutations are identified substantially simultaneously for the tumor cells.

Example 46 is directed to the system of example 43, wherein the specific primers target 10 or more loci on the genomic DNA.

Example 47 is directed to the system of example 43, wherein the specific primers target 10-500 loci on the genomic DNA.

Example 48 is directed to the system of example 43, wherein the specific primers target 500-2,000 loci on the genomic DNA.

Example 49 is directed to the system of example 43, wherein the specific primers target 2,000-100,000 loci on the genomic DNA.

Example 50 is directed to the system of example 43, wherein the lysis reagent comprises a protease.

Example 51 is directed to the system of example 43, wherein the number of tumor cells analyzed are about 10-1,000.

Example 52 is directed to the system of example 43, wherein the number of tumor cells analyzed are about 1,000-100,000

Example 53 is directed to the system of example 43, wherein the number of tumor cells analyzed are about 100,000-1,000,000

Example 54 is directed to the system of example 43, wherein the detected mutation defines at least one attribute that correlates to a known disease.

Example 55 is directed to the system of example 43, wherein presence of the mutated cell is prognostic of a disease relapse.

Example 56 is directed to the system of example 43, wherein the at least one cell originates from a patient in disease remission.

Embodiments described above illustrate but do not limit this application. While a number of exemplary aspects and embodiments have been discussed above, those of skill in the art will recognize certain modifications, permutations, additions and sub-combinations thereof. Accordingly, the scope of this disclosure is defined only by the following claims.

What is claimed is:

1. A method to detect one or more mutations in tumor cells, the method comprising:
encapsulating at least one cell and a lysis reagent in a carrier fluid to form a droplet, wherein the cell originates from a tumor and the cell comprises a genomic DNA;
lysing the cell to release the genomic DNA and thereby form a droplet containing the genomic DNA;
introducing a one or more cell identifiers and one or more primers specific to a plurality of regions of the genomic DNA; and
thermocycling the droplet to amplify the plurality of regions of genomic DNA and to incorporate the one or more cell identifiers thereby producing amplified DNA with the cell identifiers;
wherein once the cell identifier is incorporated into the amplified DNA, the amplified regions are sequenced and at least one DNA mutation is identified for the tumor cells.

2. The method of claim 1, wherein a plurality of DNA mutations are identified for the tumor cells.

3. The method of claim 1, wherein the plurality of DNA mutations are identified substantially simultaneously for the tumor cells.

4. The method of claim 1, wherein the cell identifier is an oligonucleotide that serves as a cell barcode.

5. The method of claim 1, wherein the specific primers target 5-500 loci on the genomic DNA.

6. The method of claim 1, wherein the specific primers target 500-20,000 loci on the genomic DNA.

7. The method of claim 1, wherein the lysis reagent comprises a protease.

8. The method of claim 1, wherein the number of tumor cells analyzed are about 100-1,000,000.

9. The method of claim 1, wherein the detected mutation defines at least one attribute that correlates to a known disease.

10. The method of claim 1, wherein presence of the mutated cell is prognostic of a disease relapse and wherein the at least one cell originates from a patient in disease remission.

11. A method to detect one or more mutations in cells, the method comprising:
forming a first droplet in a carrier fluid, the droplet having a tumor cell;
lysing the tumor cell and releasing the genomic DNA to provide a released genomic DNA;
forming a second droplet, the second droplet having the released genomic DNA, one or more cell identifier and one or more primers specific to a plurality of regions of the genomic DNA; and
thermocycling the second droplet to amplify the plurality of regions of genomic DNA and to incorporate the one or more cell identifiers thereby producing amplified DNA with cell identifiers;
wherein once the one or more cell identifiers are incorporated into the amplified DNA and wherein the amplified regions are sequenced and at least one DNA mutation is identified for the tumor cells.

12. The method of claim 11, wherein a plurality of DNA mutations are identified for the tumor cells.

13. The method of claim 11, wherein the plurality of DNA mutations are identified substantially simultaneously for the tumor cells.

14. The method of claim 11, wherein the specific primers target 5 or more loci on the genomic DNA.

15. The method of claim 11, wherein the specific primers target 10-2,000 loci on the genomic DNA.

16. The method of claim 11, wherein the specific primers target 2,000-100,000 loci on the genomic DNA.

17. The method of claim 11, wherein the lysis reagent comprises a protease.

18. The method of claim 11, wherein the number of tumor cells analyzed are about 1,000-1,000,000.

19. The method of claim 11, wherein the detected mutation defines at least one attribute that correlates to a known disease.

20. The method of claim 11, wherein presence of the mutated cell is prognostic of a disease relapse and wherein the at least one cell originates from a patient in disease remission.
